(19) United States
(12) Patent Application Publication
(10) Pub. No.: US 2010/0036120 A1
(43) Pub. Date: Feb. 11, 2010

(54) NOVEL METHOD FOR SYNTHESIS OF 1,4-MORPHOLINE-2,5-DIONES

(75) Inventors: Didier Bourissou, Plaisance du Touch (FR); Blaca Martin-Vaca, Toulouse (FR); Sylvie Moebs-Sanchez, Toulouse (FR); Coralie Ivens, Echirolles (FR); Roland Cherif-cheikh, Barcelona (ES); Anne-Paula De Sousa Delgado, Barclona (ES)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques (S.C.R.A.S.), Paris Cedex 16 (FR)

(21) Appl. No.: 12/162,334

(22) PCT Filed: Jan. 24, 2007
(86) PCT No.: PCT/FR2007/000135

§ 371 Date: Sep. 21, 2009

(30) Foreign Application Priority Data
Jan. 25, 2006 (FR) 06/00662

(51) Int. Cl. C07D 265/32

(52) U.S. Cl. 544/173

(57) ABSTRACT

The invention concerns a novel method for synthesis of 1,4-morpholine-2,5-diones of formula (I), wherein: R, R₁, R₂, R₃ and R₄ independently represent various radicals, by oxidizing the ketone function of a cyclic compound of formula (II).

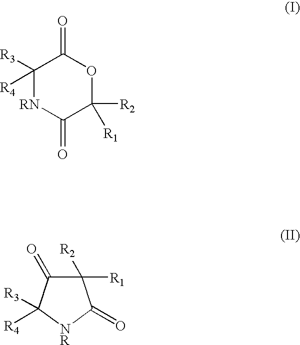

The present invention relates to a novel method for the synthesis of 1,4-morpholine-2,5-diones.

The formation of non-toxic degradation products is an essential criterion in the preparation of targeted synthetic polymers as biodegradable and biocompatible matrices for the trapping and controlled release of active ingredients. These polymers are also often formed from metabolic derivatives such as the α-hydroxylated acids or the α-amino acids. The preparation of copolymers of α-hydroxylated acids and α-amino acids, polyesteramides called polydepsipeptides, was already undertaken a few decades ago. The first syntheses of polydepsipeptides were reported at the end of the 1960s and involved the polycondensation of linear di- or tridepsipeptides (Stewart, F. H. C. Aust. J. Chem. 1969, 22, 1291; Katakai, R.; Goodman, M. Macromolecules, 1982, 15, 25). The polymers thus obtained were of low molecular weight and these multi-stage syntheses could not be developed on a larger scale. As from 1985, Feijen et al. suggested the use of cyclic didepsipeptides, the 1,4-morpholine-2,5-diones (Helder, J.; Kohn, F. E.; Sato, S.; van den Berg, J. W.; Feijen, J. Makromol. Chem., Rapid Commun. 1985, 6, 9; in't Veld, P. J. A.; Dijkstra, P. J.; Feijen, J. Makromol. Chem. 1992. 193, 2713; Dijkstra, P. J.; Feijen, J. Macromol. Symp. 2000, 153, 67). The polydepsipeptides can thus be obtained by ring-opening polymerization, as is the case with the PLGAs starting with lactide and glycolide (Dechy-Cabaret, O.; Martin-Vaca, B.; Bourissou, D., Chem. Rev. 2004, 104, 6147).

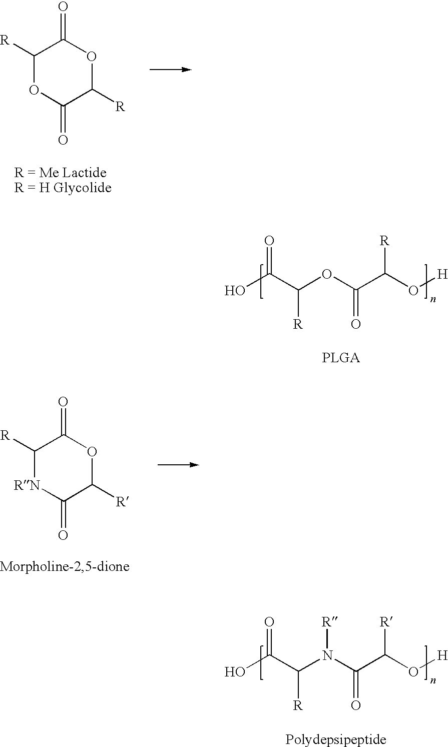

In this context, the chief benefit of the 1,4-morpholine-2,5-diones is to allow the modification of the properties of the polymers by variation of the skeleton substituents. However, there has been only a little development in this approach to date, undoubtedly due to the somewhat poor accessibility of these units.

The synthesis of the morpholine-2,5-dione precursors is generally based on a double condensation of an α-amino acid and a dihalogenated derivative (α-halogenated acid halide).

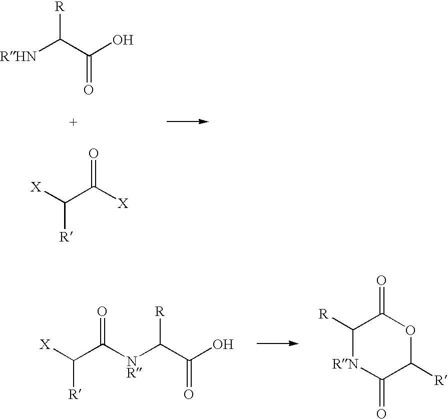

Typically, an α-amino acid and a dihalogenated derivative (α-halogenated acid halide) are condensed in a first phase under Schotten-Bauman conditions (aqueous NaOH, dioxane) in order to produce the N-(2-halogenoacyl)amino acid derivatives with yields of 50-60%. The morpholinediones are then obtained by intramolecular cyclization: either by sublimation of a mixture heated to dryness on a Celite matrix (very variable yields of 20-80%) (in't Veld, P. J. A.; Dijkstra, P. J.; van Lochem, J. H.; Feijen, J. Makromol. Chem. 1990, 191, 1813) or by treatment with triethylamine in DMF (modest yields of 3-55%) (in't Veld, P. J. A.; Dijkstra, P. J.; Feijen, J. Makromol. Chem. 1992, 193, 2713).

In practice, the isolated morpholine-2,5-dione yields are generally fairly average and the operating conditions of the cyclization stage are somewhat severe. Due to the high cis/trans inversion barrier of the amide bond, high reaction temperatures are necessary for this stage, which explains the formation of degradation products. Moreover, the key stage of intramolecular cyclization is inherently in competition with the formation of dimers and oligomers, by intermolecular rather than intramolecular reaction. The applicant has therefore envisaged a novel route for the synthesis of 1,4-morpholine-2,5-diones.

A subject of the present invention is therefore a process for the preparation of 1,4-morpholine-2,5-diones of formula (I)

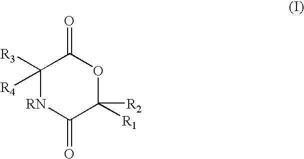

in which
R, R₁, R₂, R₃ and R₄ represent, independently, the hydrogen atom; halo; (C₂-C₆)alkenyl; (C₃-C₇)cycloalkyl; cyclohexenyl; a radical of formula —(CH₂)ₘ—V—W;
V represents a covalent bond, the oxygen or sulphur atom, or the —C(O)—O— or —NR_{N}— radical;
R_{N} represents the hydrogen atom, a (C₁-C₁₈)alkyl radical optionally substituted by one or more identical or different substituents chosen from halo and cyano; the aryl or aralkyl radical, the aryl and aralkyl radicals being optionally substituted by one or more identical or different substituents chosen from: —(CH₂)ᵣ—Y-Z, halo, nitro and cyano;
W represents the hydrogen atom; a (C₁-C₁₈)alkyl radical optionally substituted by one or more identical or different substituents chosen from halo, benzoyl, benzyloxy and cyano; (C₂-C₆)alkenyl; (C₂-C₆)alkynyl; —SiR₅R₆R₇; aryl or aralkyl, the benzoyl, benzyloxy, aryl and aralkyl radicals being optionally substituted by one or more identical or different substituents chosen from: —(CH₂)ₙ—Y-Z, halo, nitro and cyano;
R₅, R₆ and R₇ represent, independently, a (C₁-C₆)alkyl or aryl radical;
Y represents —O—, —S— or a covalent bond;
Z represents the hydrogen atom or a (C₁-C₆)alkyl radical optionally substituted by one or more identical or different halo radicals; or aralkyl;
m and n represent independently an integer from 0 to 4;

characterized in that the ketone function of a cyclic compound of formula (II)

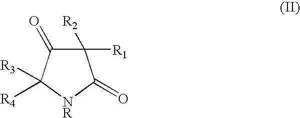

in which R, R₁, R₂, R₃ and R₄ are as defined above, is oxidized,
and in that, if desired, the compound of formula (Ia)

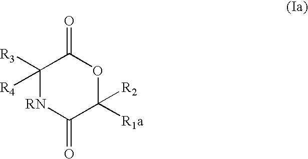

in which R, R₂, R₃ and R₄ are as defined above and R₁ₐ represents a labile group of formula —(CH₂)ₘ—V—W as defined above with m which is equal to zero and V which represents the —C(O)—O— radical, is treated with a cleavage agent in order to obtain the compound of formula (I) as defined above in which R₁ represents the hydrogen atom.

In the definitions indicated above, the expression halo represents the fluoro, chloro, bromo or iodo, preferably chloro, fluoro or bromo radical. The expression (C₁-C₆)alkyl represents an alkyl radical having 1 to 6 carbon atoms, linear or branched, such as the methyl, ethyl, propyl, isopropyl, butyl, isobutyl, sec-butyl and tert-butyl, pentyl or amyl, isopentyl, neopentyl, 2,2-dimethyl-propyl, hexyl, isohexyl or 1,2,2-trimethyl-propyl radicals. The term (C₁-C₁₈)alkyl designates an alkyl radical having 1 to 18 carbon atoms, linear or branched, such as the radicals containing 1 to 6 carbon atoms as defined above but also heptyl, octyl, 1,1,2,2-tetramethyl-propyl, 1,1,3,3-tetramethyl-butyl, nonyl, decyl, undecyl, dodecyl, tridecyl, tetradecyl, pentadecyl, hexadecyl, heptadecyl, octadecyl. By the expression alkyl substituted by at least one halo radical, is meant any linear or branched alkyl chain, containing at least one halo radical positioned along the chain such as for example —CHCl—CH₃ but also —CF₃.

In the present application also, the (CH₂)ᵢ radical (i being an integer which can represent m and n as defined above), represents a linear or branched hydrocarbon chain of i carbon atoms. Thus the —(CH₂)₃— radical can represent —CH₂—CH₂—CH₂— but also —CH(CH₃)—CH₂—, —CH₂—CH(CH₃)— or —C(CH₃)₂—.

By (C₂-C₆)alkenyl, is meant a linear or branched (alkyl)hydrocarbon radical containing 2 to 6 carbon atoms and having at least one unsaturation (double bond), such as for example vinyl, allyl, propenyl, butenyl, pentenyl or hexenyl.

By (C₂-C₆)alkynyl, is meant a linear or branched (alkyl)hydrocarbon radical containing 2 to 6 carbon atoms and having at least one double unsaturation (triple bond) such as for example an ethynyl, propargyl, butynyl or pentynyl radical.

The term (C₃-C₇)cycloalkyl designates a saturated monocyclic carbon-containing system comprising 3 to 7 carbon atoms, and preferably the cyclopropyl, cyclobutyl, cyclopentyl, cyclohexyl or cycloheptyl rings.

The expression aryl represents an aromatic radical, constituted by a condensed ring or rings, such as for example the phenyl, naphthyl, fluorenyl or anthryl radical. The term aralkyl (arylalkyl) preferably designates the radicals in which the aryl radical is as defined above and the alkyl radical is a (C₁-C₆)alkyl as defined above such as for example benzyl or phenethyl.

A more particular subject of the invention is a process as defined above, for the preparation of compound of formula (I) in which R₁ and R₂ represent, independently, halo; (C₂-C₆)alkenyl; (C₃-C₇)cycloalkyl; cyclohexenyl; or a radical of formula —(CH₂)ₘ—V—W.

A more particular subject of the invention is also a process as defined above, for the preparation of compound of formula (I) in which R₁ represents the hydrogen atom,

characterized in that the ketone function of a cyclic compound of formula (IIa)

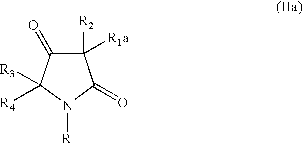

in which R, R₂, R₃ and R₄ are as defined above, and R₁ₐ represents a labile group of formula —(CH₂)ₘ—V—W as defined above with m which is equal to zero and V which represents the —C(O)—O— radical, is oxidized,
then the compound (Ia) thus obtained

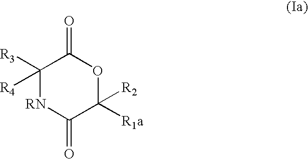

in which R, R₁ₐ, R₂, R₃ and R₄ are as defined above, is treated with a cleavage agent in order to produce the compound of formula (I) in which R₁ represents the hydrogen atom.

Preferably, the labile group that R₁ₐ represents is of formula —(CH₂)ₘ—V—W with m which is equal to zero, V represents the —C(O)—O— radical, and
W represents a (C1-C18)alkyl radical substituted by halo, benzoyl or benzyloxy; (C2-C6)alkenyl; (C2-C6)alkynyl; —SiR5R6R7; aryl or aralkyl, the benzoyl, benzyloxy, aryl and aralkyl radicals being optionally substituted by one or more identical or different substituents chosen from: —(CH2)ₙ—Y-Z, halo, nitro and cyano;
Y represents —O— or a covalent bond;
R5, R6 and R7 represent, independently, a (C1-C6)alkyl or aryl radical.

Thus, during the process of conversion of compound (II) to compound (I), the competitive dimerization and oligomerization reactions which are observed during the synthesis of morpholine-2,5-diones by condensation, are completely avoided.

For the conversion of the ketone function of compound (II) to an ester function, several types of oxidation can be implemented; the oxidation can thus take place for example in the presence of an oxidizing agent such as a peracid or a peroxide (according to the Baeyer-Villiger oxidation reaction), in the presence of a metallic catalyst (S. I. Murahashi et al., Tetrahedron Lett. 1992, 33, 7557-7760 and C. Bolm et al., Tetrahedron Lett. 1993, 34, 3405-3408) or by enzymatic route (M. D. Mihovilovic et al., Eur. J. Org. Chem. 2002, 3711-3730).

Preferably, a process according to the invention is carried out in the presence of an oxidizing agent according to the Baeyer-Villiger oxidation reaction. In this case, the oxidation reaction is very preferably carried out on the most hindered side of the ketone, such that the 1,4-morpholine-2,5-diones can be obtained highly selectively. Preferentially, the oxidizing agent is used in the presence of a catalyst.

The oxidizing agent (or oxidation agent) used for the implementation of the process according to the invention, can be a peracid or a peroxide. As examples of peracids, there can be mentioned trifluoroperacetic acid (TFPAA), peracetic acid (PAA), metachloroperbenzoic acid (m-CPBA), preferably in combination with Lewis acids (SnCl₄, Sn(OTf)₃, Re(OTf)₃) or strong acids (sulphonic acids, Nafion-H, CF₃COOH etc.). As examples of a peroxide, there can be mentioned hydrogen peroxide (H₂O₂); the hydrogen peroxide is used alone or in the presence of a catalyst which can be a Lewis acid (such as BF₃) or a metallic complex whether in homogeneous phase (Mo, Re, Pt) or in heterogeneous phase (tin zeolite, tin hydrotalcite); bis(trimethylsilyl)peroxide Me₃SiOOSiMe₃ can also be mentioned which is used in the presence of a Lewis acid (Me₃SiOTf, SnCl₄ or BF₃.OEt₂).

Preferably, the oxidizing agent is a peracid. The peracid is preferentially used in the presence of a Lewis acid or a strong acid, and more particularly in the presence of a strong acid chosen from the sulphonic acids.

The peracid is also used preferentially in the presence of a base and more particularly in the presence of an inorganic base.

Very preferentially, the peracid is metachloroperbenzoic acid (m-CPBA). The metachloroperbenzoic acid is preferentially used in the presence of trifluoromethanesulphonic acid or a hydrogen carbonate or carbonate salt.

Preferably also, the oxidizing agent is a peroxide.

Preferably also, a subject of the present invention is also a process as defined above, characterized in that said process is carried out at a temperature comprised between 20 and 80° C. in the presence of 1 to 3 molar equivalents of oxidizing agent with respect to the substrate.

Very preferentially, the process is carried out in an organic solvent, in particular chlorinated, at a substrate concentration comprised between 0.01 M and 2 M.

The abovementioned oxidizing agents are generally commercially available. The agents which are not commercially available can be synthesized according to methods known to a person skilled in the art. Thus, trifluoroperacetic acid which is not commercially available can be easily obtained by the action of hydrogen peroxide H₂O₂ on trifluoroacetic acid or anhydride CF₃CO₂H and (CF₃CO)₂O, respectively (R. Liotta et al., J. Org. Chem. 1980, 45, 2887-2890; M. Anastasia et al., J. Org. Chem. 1985, 50, 321-325; P. A. Krasutsky et al., J. Org. Chem. 2001, 66, 1701-1707). Similarly, bis(trimethylsilyl)peroxide is not commercially available but it is easily accessible starting with the complex H₂O₂-1,4-diazabicyclo[2,2,2]octane [DABCO, N(CH₂CH₂)₃N] and Me₃SiCl (P. G. Cookson et al., J. Organomet. Chem. 1975, 99, C31-C32; M. Taddei et al., Synth. Comm. 1986, 633-635).

The cyclic keto-amides of formula (II), used as precursors for the synthesis of 1,4-morpholine-2,5-diones (I) as defined above are accessible by standard methods known to a person skilled in the art (B. J. L. Royles, Chem. Rev. 1995, 95, 1981-2001 and cited references).

The reaction solvent is chosen from the organic solvents which do not interfere with the reaction. As examples of such solvents, there can be mentioned the aliphatic or aromatic chlorides (such as dichloromethane, chloroform, dichloroethane, chlorobenzene or a dichlorobenzene).

It should be noted that the R₁ and R₂ radicals of general formula (I) as defined in the present application, are equivalent and therefore interchangeable.

In the case where R₁ represents the hydrogen atom, compound (I) can be also obtained from the morpholidione of formula (Ia)

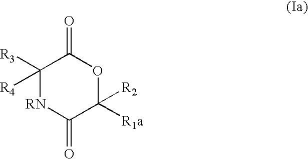

in which R₁ₐ is a labile group of formula —C(O)—O—W, after cleavage of this labile group R₁ₐ.

Various reagents and conditions, well known to a person skilled in the art and described in detail in various works (Wuts, P. G. M.; Greene, T. W.; Protective Groups in Organic Synthesis, 4^{th} edition, 2006, Wiley Interscience; Kocienski, P. J. Protecting Groups, 3^{rd} Edition, 2003, Georg Thieme Verlag) allow the cleavage of the R₁ₐ group as defined above and lead, after decarboxylation, to compound (I) in which the R₁ group represents the hydrogen atom. As examples of R₁ₐ labile groups, there can be mentioned the benzyloxycarbonyl, (benzyloxy)methoxycarbonyl, (benzoyl)methoxycarbonyl, allyloxycarbonyl, propargyloxycarbonyl, trimethylsilyloxycarbonyl groups. With these groups, catalytic hydrogenation is a cleavage method of choice.

A more particular subject of the present invention is also a process as defined above, characterized in that R represents the hydrogen atom or a radical of formula —(CH₂)ₘ—V—W with V which represents a covalent bond or the —C(O)—O— radical and W an optionally substituted aralkyl radical; R₁, R₂, R₃ and R₄ represent, independently, the hydrogen atom or a radical of formula —(CH₂)ₘ—V—W with V which represents a covalent bond and W a (C₁-C₆)alkyl radical.

A more particular subject of the present invention is also a process as defined above, characterized in that R represents the hydrogen atom or an optionally substituted aralkyl radical.

A more particular subject of the present invention is also a process as defined above, characterized in that the term aryl of the aryl and aralkyl radicals is the phenyl radical and m is equal to zero or one.

A more particular subject of the present invention is also a process as defined above, characterized in that R represents the hydrogen atom or the benzyl radical, R₁ and R₂, represent, independently, the hydrogen atom or the methyl or ethyl radical, and R₃ and R₄ represent, independently, the hydrogen atom or a methyl radical.

A subject of the present invention is also a process for the preparation of 1,4-morpholine-2,5-diones of formula (I)

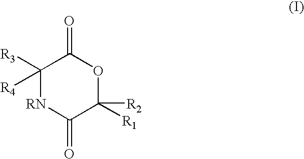

in which
R, R1, R2, R3 and R4 represent, independently, the hydrogen atom; halo; (C2-C6)alkenyl; (C3-C7)cycloalkyl; cyclohexenyl; a radical of formula —(CH2)ₘ—V—W;
V represents a covalent bond, the oxygen or sulphur atom, or the —C(O)—O— or —NRN— radical;
RN and W represent, independently, the hydrogen atom, a (C1-C18)alkyl radical optionally substituted by one or more identical or different substituents chosen from halo and cyano; the aryl or aralkyl radical, the aryl and aralkyl radicals being optionally substituted by one or more identical or different substituents chosen from: —(CH2)n-Y-Z, halo, nitro and cyano;
Y represents —O—, —S— or a covalent bond;
Z represents the hydrogen atom or a (C1-C6)alkyl radical optionally substituted by one or more identical or different halo radicals; or aralkyl;
m and n represent independently an integer from 0 to 4;
   by oxidation of the ketone function of a cyclic compound of formula (II)

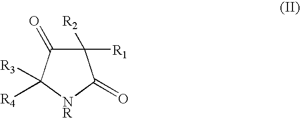

in which R, R₁, R₂, R₃ and R₄ are as defined above.

A subject of the present invention is also compounds of formula (I) and in particular the compounds (I) as obtained according to the process defined above.

A subject of the present invention is also compounds of formula (Ib)

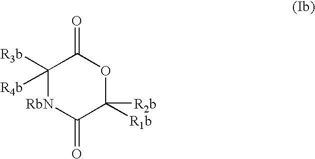

which can be obtained according to the process defined above, and characterized in that
R_{b} represents an arylalkyl radical;
R_{1b}, R_{3b} and R_{4b} represent, independently, the hydrogen atom; halo; (C₂-C₆)alkenyl; (C₃-C₇)cycloalkyl; cyclohexenyl; or a radical of formula —(CH₂)ₘ—V—W;
R_{2b} represents halo; (C₂-C₆)alkenyl; (C₃-C₇)cycloalkyl; cyclohexenyl; a radical of formula —(CH₂)ₘ—V—W;
V represents a covalent bond, the oxygen or sulphur atom, the —C(O)—O— or —NR_{N}— radical;
R_{N} represents the hydrogen atom; a (C₁-C₁₈)alkyl radical optionally substituted by one or more identical or different substituents chosen from halo and cyano; the aryl or aralkyl radical, the aryl and aralkyl radicals being optionally substituted by one or more identical or different substituents chosen from: —(CH₂)ₙ—Y-Z, halo, nitro and cyano;
W represents the hydrogen atom, a (C₁-C₁₈)alkyl radical optionally substituted by one or more identical or different substituents chosen from halo, benzoyl, benzyloxy and cyano; (C₂-C₆)alkenyl; (C₂-C₆)alkynyl; —SiR₅R₆R₇; the aryl or aralkyl radical, the benzoyl, benzyloxy, aryl and aralkyl radicals being optionally substituted by one or more identical or different substituents chosen from: —(CH₂)ₙ—Y-Z, halo, nitro and cyano;
R₅, R₆ and R₇ represent, independently, a (C₁-C₆)alkyl or aryl radical;
Y represents —O—, —S— or a covalent bond;
Z represents the hydrogen atom or a (C₁-C₆)alkyl radical optionally substituted by one or more identical or different halo radicals; or aralkyl;
m and n represent independently an integer from 0 to 4;
   it being understood that
when W represents the —SiR₅R₆R₇ radical, then V represents the —C(O)—O— radical and m is equal to zero; and
when R_{1b} represents the hydrogen atom and R_{2b} the radical of formula —(CH₂)ₘ—V—W with m which is equal to 1 and V which represents the —C(O)—O— radical, then W does not represent the hydrogen atom.

A more particular subject of the present invention is compounds of formula (I_{b}) as defined above, and characterized in that R_{1b} represents the hydrogen atom or a radical of formula —(CH₂)ₘ—V—W with V which represents a covalent bond or the —C(O)—O— radical; and R_{2b} represents a radical of formula —(CH₂)ₘ—V—W with V which represents a covalent bond or the —C(O)—O— radical. Preferably, R_{1b} represents the hydrogen atom or a (C₁-C₆)alkyl radical and R_{2b} represents a (C₁-C₆)alkyl radical.

Preferably also, the compounds of formula (I_{b}) as defined above are such that the term aryl of the aryl and aralkyl radicals is the phenyl radical.

A more particular subject of the present invention is also compounds of formula (I_{b}) as defined above, and characterized in that R_{b} represents the optionally substituted benzyl radical.

A more particular subject of the present invention is also compounds of formula (I_{b}) as defined above, and characterized in that R_{3b} and R_{4b} represent, independently, the hydrogen atom or a (C₁-C₆)alkyl radical. Preferably, R_{3b} represents the hydrogen atom, and R_{4b} represents the hydrogen atom or a (C₁-C₆)alkyl radical.

A more particular subject of the present invention is also the compounds of formula (I) as defined above, and characterized in that R_{1b} represents the hydrogen atom, the methyl, carboxy or benzyloxycarbonyl radical, R_{2b} a methyl radical, R_{3b} and R_{4b} the hydrogen atom, and R_{b} the benzyl radical.

A subject of the present invention is also the use of the compounds of formula (I) or (I_{b}) and in particular the compounds (I) or (I_{b}) as obtained according to the process defined above, for the preparation of polydepsipeptides.

### EXPERIMENTAL PART

### Example 1

### Synthesis of 4-benzyl-6,6-dimethyl-1,4-morpholine-2,5-dione

### STAGE 1: synthesis of the precursor (II): 1-benzyl-3,3-dimethylpyrrolidine-2,4-dione

The synthesis of compound (II) can take place according to two reaction diagrams described below:

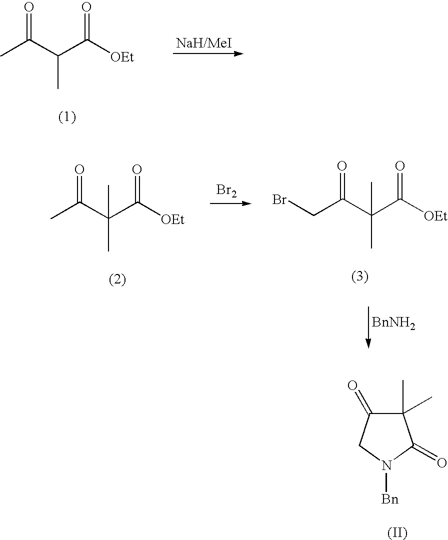

(II) can be obtained by a 3-stage route starting with (1). The formation of compound (2) from compound (1) can take place according to H. C. Brown et al., J. Am. Chem. Soc. 1988, 110, 1539-1546. The synthesis stage of compound (3) can take place according to M. Conrad et al., Ber. 1898, 31, 2726-2731. Finally the final cyclization stage takes place spontaneously after treatment of (3) with benzylamine (2.2 equiv.) in tetrahydrofuran according to Falk, H. et al. Monatsch. Chem., GE 113, 1982, 11, 1329-1348. 1-benzyl-3,3-dimethylpyrrolidine-2,4-dione (II) is obtained with a yield of 42% from (1). The product (II) is characterized by NMR (CDCl₃+TMS) ¹H [1.26 (s, 6H, —CH₃); 3.70 (s, 2H, —CH₂); 4.63 (s, 2H, CH₂); 7.24-7.38 (m, 5H, arom H)] and ¹³C [20.5 (—CH₃); 45.8 (CH₂); 47.1 (Cq); 53.6 (CH₂); 128.0; 128.2; 129.0 and 135.3 (arom C), 175.6 (—CON); 210.3 (—CO)]. MS (EI) 217[M M]⁺, melting point 61° C.

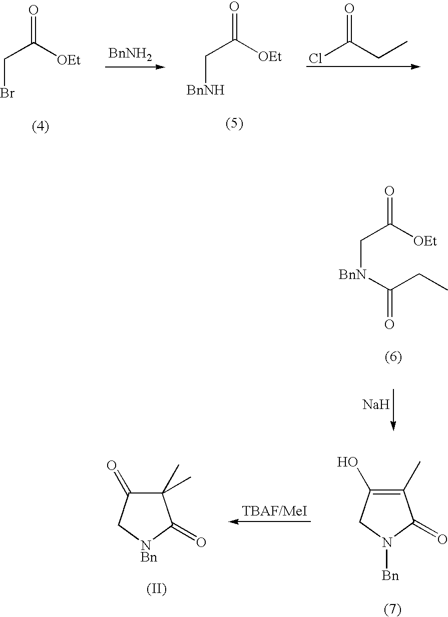

(II) can also be obtained from 3-methyl tetramic acid (7). The formation of compound (7) from compound (4) can take place according to Koech, P. et al., Org. Lett. 2004, 6, 691-694 with a yield of 98% over the three stages. The final stage of formation of compound (II) can take place according to Page, P. C. B. et al., Org. Lett. 2003, 5, 353-355 with a yield of 78% of isolated product.

### STAGE 2: oxidation of the keto-amide (II) to 4-benzyl-6,6-dimethyl-1,4-morpholine-2,5-dione

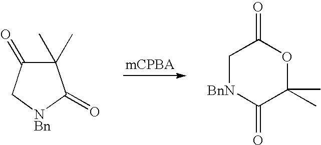

Conditions 1:

A solution of 1.09 g of cyclic keto-amide (5 mmol), 1.55 g of metachloroperbenzoic acid (1.8 eq.) and 2.73 g of sodium bicarbonate (6.5 eq.) in 100 mL of dichloromethane is stirred at ambient temperature for twenty-six hours. ¹H NMR testing of an aliquot of the reaction medium reveals the formation of a very major proportion of 4-benzyl-6,6-dimethyl-1,4-morpholine-2,5-dione (spectroscopic yield: 90%).

Conditions 2:

A solution of 9.0 g of ketoamide precursor (41.4 mmol) and 9.0 g of mCPBA (1.2 eq.) in 40 mL of anhydrous dichloromethane under an inert atmosphere is stirred under reflux for twenty hours. The ¹H NMR testing of an aliquot of the reaction medium reveals the virtually quantitative formation of the ring with six members (spectroscopic yield >99%). The mixture is washed with an aqueous solution of sodium thiosulphate (5%), with an aqueous solution of sodium hydrogen carbonate (5%) then with a saturated aqueous solution of sodium chloride. The organic phase is dried over magnesium sulphate, concentrated then dried under vacuum. The residual brown oil is recrystallized from a dichloromethane/diethyl ether mixture in order to produce 665 mg of analytically pure white crystals of 4-benzyl-6,6-dimethyl-1,4-morpholine-2,5-dione (57% yield of isolated product). The product is characterized by NMR (CDCl₃+TMS) ¹H [1.65 (s, 6H, —CH₃); 4.01 (s, 2H, —CH₂CO); 4.61 (s, 2H, —CH₂Ph); 7.28-7.33 (m, 5H, arom H)] and ¹³C [25.7 (—CH₃); 47.7 (—CH₂CO); 49.6 (NCH₂Ph); 82.1 (Cq); 128.2, 128.4 and 129.1 (arom CH); 134.8 (Carom Cq), 164.9 (—COO); 168.2 (—CON)]. MS (EI) 233 [M M]⁺, melting point 94.5° C. and elementary analysis Calculated C, 66.94; H, 6.48; N, 6.00. Found C, 66.92; H, 6.34; N, 5.94.

Conditions 3:

A solution of 0.20 g of cyclic keto-amide (0.92 mmol), 0.32 g of metachloroperbenzoic acid (2.0 eq.) and 16 [M2 L of trifluoromethanesulphonic acid (0.2 eq.) in 1.0 mL of dichloromethane is stirred at ambient temperature for twenty-four hours. ¹H NMR testing of an aliquot of the reaction medium reveals the formation of a very major proportion of 4-benzyl-6,6-dimethyl-1,4-morpholine-2,5-dione (spectroscopic yield >90%).

### Example 2

### Synthesis of 4-benzyl-3-methyl-1,4-morpholine-2,5-dione

### STAGE 1: synthesis of precursor (II): 1-benzyl-3-carboxybenzyl-3-methylpyrrolidine-2,4-dione

(II) is obtained according to a 4-stage route as described by Page, P. C. B. et al. Org. Lett. 2003, 5, 353-355, with a yield of 44% from (4).

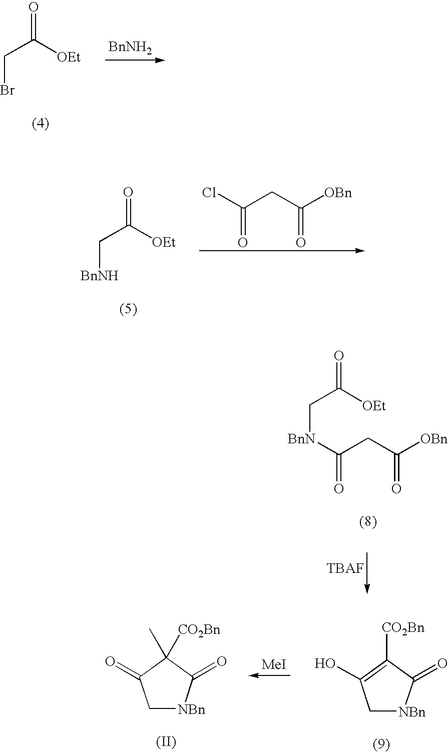

### STAGE 2: oxidation of ketoamide (II) to 4-benzyl-6-carboxybenzyl-6-methyl-1,4-morpholine-2,5-dione (2a)

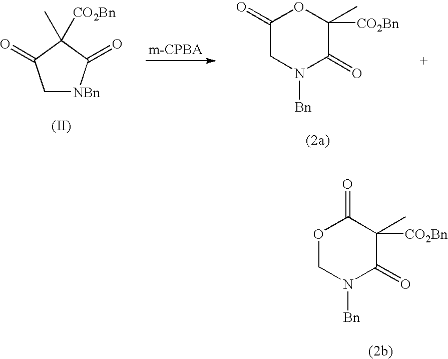

A solution of 1.02 g of cyclic keto-amide (II) (3 mmol) and 1.03 g of metachloroperbenzoic acid (1.3 equiv.) in 3 mL of dichloromethane is heated to reflux for 4 days. ¹H NMR analysis reveals the complete conversion of the ring with 5 members and the formation of a major proportion of N-benzyl-6-carboxybenzyl-6-methyl-1,4-morpholine-2,5-dione (2a) and the regioisomer N-5-benzyl-3-carboxybenzyl-3-methyl-1,5-morpholine-2,4-dione (2b). After returning to ambient temperature, the medium is treated with 2 g of Amberlyst® A21 basic resin (4.6 eq. of base/g of resin) for two hours then filtered and evaporated. ¹H NMR analysis confirms the elimination of the acids. A residual yellow oil is obtained with 92% crude yield (NMR ratio (2a)/(2b) 1.8/1). The two regioisomers can be obtained analytically pure after chromatography on silica (eluent petroleum ether/ethyl acetate 2/1) with 41% yield of (2a) and 25% of (2b). The two products are characterized by ¹H, ¹³C NMR, MS (EI), IR.

Characterization of (2a):

NMR (CDCl₃) ¹H (300 MHz) [1.92 (s, 3H, CH₃); 3.87 and 3.98 (2d, 2H, J 18.0 Hz, NCH₂CO); 4.33 and 4.69 (2d, 2H, J 14.0 Hz, NCH₂Ph); 5.20 and 5.28 (2d, 2H, J 12.0 Hz, CO₂CH₂); 7.10-7.13 and 7.29-7.37 (2m, 2H and 8H, Ar—H)] and ¹³C (75 MHz) [20.6 (CH₃); 48.1 (NCH₂CO); 50.0 (NCH₂Ph); 68.8 (COOCH₂Ph); 83.2 (Cq); 128.1; 128.2; 128.4; 128.8; 128.9; 129.1; 134.2; 162.4 (NCO); 164.3 (COO); 167.2 (COOCH₂Ph)]. IR(CHCl₃) 1775, 1750, 1687 cm⁻¹, MS (EI) 353 [M M]⁺ and elementary analysis Calculated C, 67.98; H, 5.42; N, 3.96. Found C, 67.65; H, 5.20; N, 3.96.

Characterization of (2b):

NMR (CDCl₃) ¹H (300 MHz) [1.81 (s, 3H, CH₃); 4.52 and 4.81 (2d, 2H, J 15.0 Hz, NCH₂Ph); 4.92 and 5.05 (2d, 2H, J 10.5 Hz, OCH₂); 5.23 (s, 2H, CO₂CH₂); 7.15-7.19 and 7.30-7.37 (2m, 2H and 8H, Ar—H)] and ¹³C (75 MHz) [17.7 (CH₃); 46.1 (Cq), 49.1 (NCH₂Ph); 68.8 (COOCH₂Ph); 74.4 (OCH₂); 128.1; 128.2; 128.4; 128.8; 128.9; 129.1; 134.7; 163.7 (NCO); 165.5 (COOCH₂Ph); 165.9 (COO)]. IR(CHCl₃) 1778, 1739, 1699 cm⁻¹, MS (EI) 353 [M M]⁺.

### STAGE 3: synthesis of 4-benzyl-6-methyl-1,4-morpholine-2,5-dione

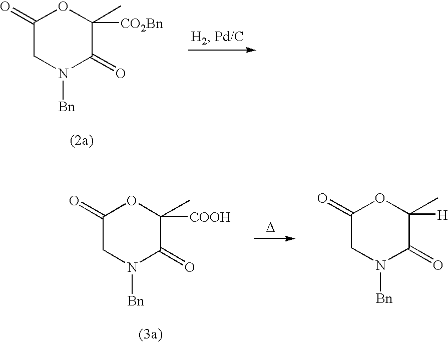

(2a) then undergoes one-pot conversion to 4-benzyl-6-methyl-1,4-morpholine-2,5-dione. A solution of (2a) in 30 mL of toluene is stirred under atmospheric pressure of hydrogen in the presence of 10% Pd/C at ambient temperature for twelve hours. ¹H NMR analysis reveals the complete conversion of (2a) to (3a): NMR (CD₃OD) ¹H (300 MHz) [1.83 (s, 3H, CH₃); 4.10 (s, 2H, OCH₂); 4.36 and 4.90 (2d, 2H, J 14.4 Hz, NCH₂)] and ¹³C (75 MHz) [20.9 (CH₃); 49.8 (OCH₂); 50.7 (NCH₂Ph); 84.6 (Cq); 129.0; 129.2; 130.0; 136.5; 165.0 (NCO); 166.8 (COO) and 170.1 (COOH)]. IR(KBr) 2920 (COOH), 1769, 1642 cm⁻¹, MS (EI) 262 [M M]⁺. After filtration, the mixture is heated to reflux for 15 minutes then evaporated to dryness. Recrystallization from a dichloromethane/diethyl ether mixture makes it possible to obtain analytically pure white crystals of 4-benzyl-6-methyl-1,4-morpholine-2,5-dione with a 64% yield. The product was characterized by NMR (CDCl₃) ¹H (300 MHz)[7.31-7.14 (m, 5H, Ar—H), 4.85 (q, 1H, J 7.2 Hz, CH), 4.56 and 4.48 (2d, 2H, J 14.4 Hz, CH₂), 3.96 and 3.88 (2d, 2H, J 18.0 Hz, NCH₂), 1.57 (d, 3H, J 6.9 Hz, CH₃)] and ¹³C (75 MHz) [166.1 (NCO), 165.1 (COO), 134.6, 129.1, 128.4, 128.2, 75.0 (CH), 49.4 (NCH₂Ph), 47.3 (CH₂), 17.4 (CH₃)]. IR(KBr) 1759, 1663 cm⁻¹; MS (EI) 219 [M]⁺; melting point 95.1-95.3° C. and elementary analysis: Calculated C, 65.74; H, 5.98; N, 6.39. Found C, 65.59; H, 6.01; N, 6.35.

1. A process for the preparation of 1,4-morpholine-2,5-diones of formula (I) 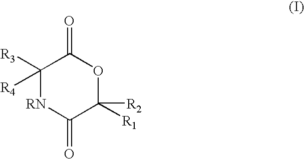
in which R, R₁, R₂, R₃ and R₄ represent, independently, the hydrogen atom; halo; (C₂-C₆)alkenyl; (C₃-C₇)cycloalkyl; cyclohexenyl; a radical of formula —(CH₂)ₘ—V—W;
V represents a covalent bond, the oxygen or sulphur atom, the —C(O)—O— or —NR_{N}— radical;
R_{N} represents the hydrogen atom; a (C₁-C₁₈)alkyl radical optionally substituted by one or more identical or different substituents chosen from halo and cyano; the aryl or aralkyl radical, the aryl and aralkyl radicals being optionally substituted by one or more identical or different substituents chosen from: —(CH₂)ₙ—Y-Z, halo, nitro and cyano;
W represents the hydrogen atom; a (C₁-C₁₈)alkyl radical optionally substituted by one or more identical or different substituents chosen from halo, benzoyl, benzyloxy and cyano;
(C₂-C₆)alkenyl; (C₂-C₆)alkynyl; —SiR₅R₆R₇; aryl or aralkyl, the benzoyl, benzyloxy, aryl and aralkyl radicals being optionally substituted by one or more identical or different substituents chosen from: —(CH₂), —Y-Z, halo, nitro and cyano;
R₅, R₆ and R₇ represent, independently, a (C₁-C₆)alkyl or aryl radical;
Y represents —O—, —S— or a covalent bond;
Z represents the hydrogen atom or a (C₁-C₆)alkyl radical optionally substituted by one or more identical or different halo radicals; or aralkyl;
m and n represent independently an integer from 0 to 4;
comprising oxidizing the ketone function of a cyclic compound of formula (II) 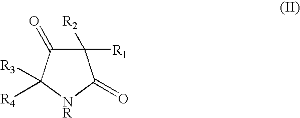
in which R, R₁, R₂, R₃ and R₄ are as defined above; and
optionally treating the compound of formula (Ia) 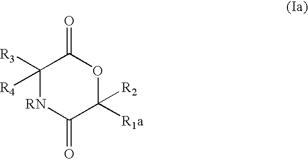
with a cleavage agent in order to obtain the compound of formula (I) in which R₁ represents the hydrogen atom, such that, with respect to the compound of formula (Ia), R, R₂, R₃ and R₄ are as defined above and R₁ₐ represents a labile group of formula —(CH₂)ₘ—V—W as defined above with m which is equal to zero and V which represents the —C(O)—O— radical.

2. The process according to claim 1, for the preparation of the compound of formula (I) in which R₁ and R₂ represent, independently, halo; (C₂-C₆)alkenyl; (C₃-C₇)cycloalkyl; cyclohexenyl; or a radical of formula —(CH₂)ₘ—V—W.

3. The process according to claim 1, for the preparation of the compound of formula (I) in which R₁ represents the hydrogen atom,
comprising oxidizing the ketone function of a cyclic compound of formula (IIa) 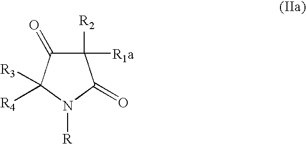
in which R, R₂, R₃ and R₄ are as defined in claim 1, and R₁ₐ represents a labile group of formula —(CH₂)ₘ—V—W as defined in claim 1 with m which is equal to zero and V which represents the —C(O)—O— radical,
treating the compound (Ia) thus obtained 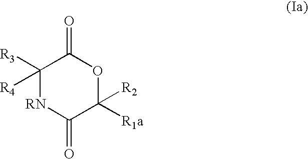
with a cleavage agent in order to produce the compound of formula (I) in which R₁ represents hydrogen, such that, with respect to the compound of formula (Ia), R, R₁ₐ, R₂, R₃ and R₄ are as defined above.

4. The process according to claim 1, wherein the labile group that R₁ₐ represents is of formula —(CH₂)ₘ—V—W with m which is equal to zero, V represents the —C(O)—O— radical, and
W represents a (C₁-C₁₈)alkyl radical substituted by halo, benzoyl or benzyloxy; (C₂-C₆)alkenyl; (C₂-C₆)alkynyl; —SiR₅R₆R₇; aryl or aralkyl, the benzoyl, benzyloxy, aryl and aralkyl radicals being optionally substituted by one or more identical or different substituents chosen from: —(CH₂)ₙ—Y-Z, halo, nitro and cyano;
Y represents —O— or a covalent bond; and
R₅, R₆ and R₇ represent, independently, a (C₁-C₆)alkyl or aryl radical.

5. The process according to claim 1, wherein the process is carried out in the presence of an oxidizing agent.

6. The process according to claim 5, wherein the oxidizing agent is used in the presence of a catalyst.

7. The process according to claim 5, wherein the oxidizing agent is a peracid or a peroxide.

8. The process according to claim 5, wherein the oxidizing agent is a peracid.

9. The process according to claim 8, wherein the oxidizing agent is used in the presence of a Lewis acid or a strong acid.

10. The process according to claim 9, wherein the oxidizing agent is used in the presence of a strong acid chosen from the sulphonic acids.

11. The process according to claim 8, wherein the oxidizing agent is used in the presence of a base.

12. The process according to claim 11, wherein the oxidizing agent is used in the presence of an inorganic base.

13. The process according to claim 8, wherein the oxidizing agent is metachloroperbenzoic acid.

14. The process according to claim 13, wherein the oxidizing agent is used in the presence of trifluoromethanesulphonic acid.

15. The process according to claim 13, wherein the oxidizing agent is used in the presence of a hydrogen carbonate or carbonate salt.

16. The process according to claim 5, wherein the oxidizing agent is a peroxide.

17. The process according to claim 1, wherein R represents the hydrogen atom or a radical of formula —(CH₂)ₘ—V—W with V which represents a covalent bond or the —C(O)—O— radical and W an optionally substituted aralkyl radical; R₁, R₂, R₃ and R₄ represent, independently, the hydrogen atom or a radical of formula —(CH₂)ₘ—V—W with V which represents a covalent bond and W a (C₁-C₆)alkyl radical.

18. The process according to claim 1, wherein R represents the hydrogen atom or an optionally substituted aralkyl radical.

19. The process according to claim 1, wherein the term aryl of the aryl and aralkyl radicals is the phenyl radical and m is equal to zero or one.

20. The process according to claim 1, wherein R represents the hydrogen atom or the benzyl radical, R₁ and R₂, represent, independently, the hydrogen atom or the methyl or ethyl radical, and R₃ and R₄ represent, independently, the hydrogen atom or a methyl radical.

21. The process according to claim 1, wherein said process is carried out at a temperature between 20 and 80° C. in the presence of 1 to 3 molar equivalents of oxidizing agent with respect to the substrate.

22. The process according to claim 1, wherein said process is carried out in an organic solvent, at a substrate concentration between 0.01 M and 2 M.

23. A compound according to formula (Ib) 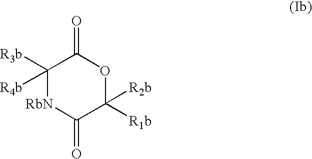
in which
R_{b} represents an arylalkyl radical;
R_{1b}, R_{3b} and R_{4b} represent, independently, the hydrogen atom; halo; (C₂-C₆)alkenyl; (C₃-C₇)cycloalkyl; cyclohexenyl; or a radical of formula —(CH₂)ₘ—V—W;
R_{2b} represents halo; (C₂-C₆)alkenyl; (C₃-C₇)cycloalkyl; cyclohexenyl; a radical of formula —(CH₂)ₘ—V—W;
V represents a covalent bond, the oxygen or sulphur atom, the —C(O)—O— or —NR_{N}— radical;
R_{N} represents the hydrogen atom; a (C₁-C₁₈)alkyl radical optionally substituted by one or more identical or different substituents chosen from halo and cyano; the aryl or aralkyl radical, the aryl and aralkyl radicals being optionally substituted by one or more identical or different substituents chosen from: —(CH₂)ₙ—Y-Z, halo, nitro and cyano;
W represents the hydrogen atom, a (C₁-C₁₈)alkyl radical optionally substituted by one or more identical or different substituents chosen from halo, benzoyl, benzyloxy and cyano;
(C₂-C₆)alkenyl; (C₂-C₆)alkynyl; —SiR₅R₆R₇; the aryl or aralkyl radical, the benzoyl, benzyloxy, aryl and aralkyl radicals being optionally substituted by one or more identical or different substituents chosen from: —(CH₂), —Y-Z, halo, nitro and cyano;
R₅, R₆ and R₇ represent, independently, a (C₁-C₆)alkyl or aryl radical;
Y represents —O—, —S— or a covalent bond;
Z represents the hydrogen atom or a (C₁-C₆)alkyl radical optionally substituted by one or more identical or different halo radicals; or aralkyl;
m and n represent independently an integer from 0 to 4;
it being understood that
when W represents the —SiR₅R₆R₇ radical, then V represents the —C(O)—O— radical and m is equal to zero; and
when R_{1b} represents the hydrogen atom and R_{2b} the radical of formula —(CH₂)ₘ—V—W with m which is equal to 1 and V which represents the —C(O)—O— radical, then W does not represent the hydrogen atom.

24. The compounds according to claim 23, wherein R_{1b} represents the hydrogen atom or a radical of formula —(CH₂)ₘ—V—W with V which represents a covalent bond or the —C(O)—O— radical; and R_{2b} represents a radical of formula —(CH₂)ₘ—V—W with V which represents a covalent bond or the —C(O)—O— radical.

25. The compounds according to claim 23, wherein R_{1b} represents the hydrogen atom or a (C₁-C₆)alkyl radical and R_{2b} represents a (C₁-C₆)alkyl radical.

26. The compounds according to claim 23, wherein the term aryl of the aryl and aralkyl radicals is the phenyl radical.

27. The compound according to claim 23, wherein R_{b} represents the optionally substituted benzyl radical.

28. The compounds according to claim 23, wherein R_{3b} and R_{4b} represent, independently, the hydrogen atom or a (C₁-C₆)alkyl radical.

29. The compounds according to claim 23, wherein R_{3b} represents the hydrogen atom and R_{4b} represents the hydrogen atom or a (C₁-C₆)alkyl radical.

30. The compounds according to claim 23, wherein R_{1b} represents the hydrogen atom, the methyl, carboxy or benzyloxycarbonyl radical, R_{2b} a methyl radical, R_{3b} and R_{4b} the hydrogen atom, and R_{b} the benzyl radical.

31. The process according to claim 22, wherein the organic solvent is a chlorinated organic solvent.
